(19) United States
(12) Patent Application Publication
(10) Pub. No.: US 2023/0053812 A1
(43) Pub. Date: Feb. 23, 2023

(54) STABLE PEPTIDE FORMULATIONS FOR ORAL USE

(71) Applicant: Aurobindo Pharma Ltd, (US)

(72) Inventors: Saravanan KANNUSAMY, Hyderabad (IN); Prabhakaran CHAKKIRALA, Hyderabad (IN); Raghavendra Bharat GUNDA, Hyderabad (IN); Nagaprasad VISHNUBHOTLA, Hyderabad (IN); Meenakshisunderam SIVAKUMARAN, Hyderabad (IN)

(21) Appl. No.: 17/875,117

(22) Filed: Jul. 27, 2022

(30) Foreign Application Priority Data
Jul. 27, 2021 (IN) 202141033678

(51) Int. Cl. A61K 38/26; A61K 9/20; A61K 38/31; A61K 9/48; A61K 9/00; A61K 47/22; A61K 38/28; A61K 38/22; A61K 38/095; A61K 38/23; A61K 38/29; A61K 31/401

(52) U.S. Cl. CPC A61K 38/26; A61K 9/2013; A61K 9/2027; A61K 9/2054; A61K 38/31; A61K 9/4858; A61K 9/4866; A61K 9/009; A61K 47/22; A61K 38/28; A61K 38/22; A61K 38/095; A61K 38/23; A61K 38/29; A61K 31/401

(57) ABSTRACT

The invention relates to stable pharmaceutical formulation useful for the oral administration comprising a peptide or protein drug and a permeation enhancer. In particular, the invention relates to stabilized pharmaceutical formulations comprising GLP-1 receptor agonist such as Semaglutide and a permeation enhancer and its process for preparation.

### FIELD OF THE INVENTION

The present invention relates to stable pharmaceutical formulation useful for the oral administration comprising peptide or protein drug and a permeation enhancer. The present invention also provides stabilized pharmaceutical formulation useful for the oral administration comprising a glucagon-like peptide-1 (GLP-1) receptor agonist and a permeation enhancer, wherein the dosage forms comprising the said stable formulations preferably release of the drug rapidly and is bioavailable. In particular, the present invention provides stabilized pharmaceutical formulations comprising Semaglutide and a permeation enhancer and its process for preparation. Oral administration of the formulations can be used for treatment of type 2 diabetes as well as a variety of other conditions.

### BACKGROUND OF THE INVENTION

Delivering proteins and peptides by the oral route is extremely challenging due to the nature of digestive system which is designed to breakdown the polypeptides into amino acids prior to absorption. The low bioavailability of drugs remains to be an active area of research and several sites in the gastrointestinal tract have been investigated by researchers, but no major breakthrough with broad applicability to diverse proteins and peptides has been achieved.

Over the past several decades, continuous efforts have been made to improve the treatment of diabetes mellitus. Approximately 90% of people with diabetes have type 2 diabetes, also known as non-insulin dependent diabetes mellitus (NIDDM). Type 2 diabetics generally still make insulin, but the insulin cannot be used effectively by the body's cells. This is primarily because the amount of insulin produced in response to rising blood sugar levels is not sufficient to allow cells to efficiently take up glucose and thus, reduce blood sugar levels.

A large body of preclinical and clinical research data suggests that GLP-1 compounds show great promise as a treatment for type 2 diabetes and other conditions. GLP-1 induces numerous biological effects such as stimulating insulin secretion, inhibiting glucagon secretion, inhibiting gastric emptying, enhancing glucose utilization, and inducing weight loss. Further, pre-clinical studies suggest that GLP-1 may also act to prevent the βcell deterioration that occurs as the disease progresses. Perhaps the most salient characteristic of GLP-1 is its ability to stimulate insulin secretion without the associated risk of hypoglycemia that is seen when using insulin therapy or some types of oral therapies that act by increasing insulin expression.

Semaglutide is a recombinant long acting GLP-1 receptor agonist. The GLP-1 analogue is acylated at lysine 26 with a fatty diacid moiety and has two amino acid substitutions (Ala8 to Aib8 (2-aminoisobutyric acid), Lys34 to Arg34) compared to human GLP-1.

Semaglutide is produced using recombinant DNA technology in yeast (Saccharomyces cerevisiae) and chemical modification. Compared to human GLP-1, semaglutide has a prolonged half-life and the principal mechanism of protraction is albumin binding, which results in decreased renal clearance and protection from metabolic degradation. Furthermore, semaglutide is stabilised against degradation by the dipeptidyl peptidase 4 (DPP-4) enzyme by the amino acid substitution Ala8 to Aib8.

The theoretical relative monoisotopic molecular mass of semaglutide is 4111.115 and the theoretical average molecular weight is 4113.58 g/mol. Semaglutide structurally represented as:

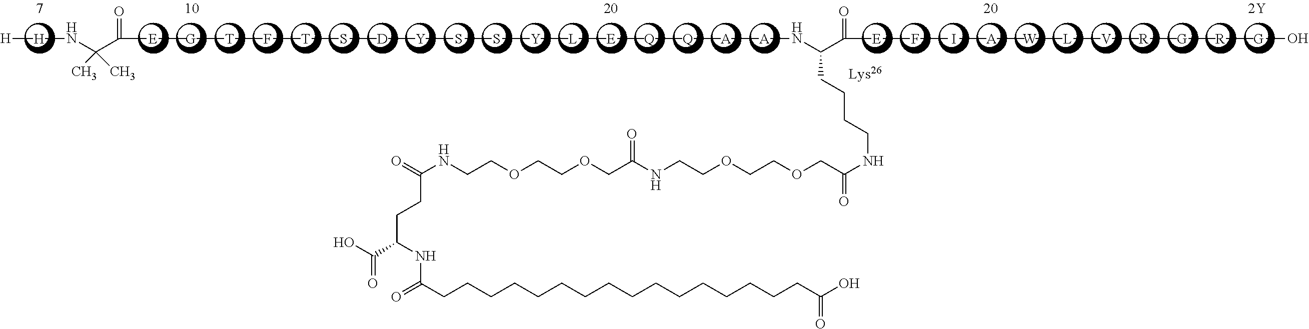

Semaglutide is indicated as an adjunct to diet and exercise to improve glycemic control in adults with type 2 diabetes mellitus. It works by reducing blood glucose through a mechanism where it stimulates insulin secretion and lowers glucagon secretion, both in a glucose-dependent manner. Thus, when blood glucose is high, insulin secretion is stimulated and glucagon secretion is inhibited. The mechanism of blood glucose lowering also involves a minor delay in gastric emptying in the early postprandial phase.

Currently, Semaglutide (3 mg, 7 mg, and 14 mg) is marketed under the brand name “RYBELSUS” in the United States by Novo Nordisk Inc and is supplied as tablets for oral administration.

Several attempts have been made in the prior art to provide optimal and stable solid dosage form comprising GLP-1 compound. Beglinger et al. (Clin Pharmacol Ther 2008 October; 84(4):468-74) teaches tablets comprising 0.5, 1.0, 2.0 or 4 mg GLP-1 peptide (7-36 amide) and 200 mg sodium N-(8-(2-hydroxybenzoyl)amino)caprylate (SNAC). Steinert et al. (Am J Clin Nutr, October 2010; 92: 810-817) discloses oral administration of a tablet comprising GLP-1 (7-36) amide and 150 mg sodium N-(8-(2-hydroxybenzoyl)amino)caprylate (SNAC).

U.S. Pat. No. 8,129,343 discloses semaglutide specifically and its composition and/or method of use. U.S. Pat. No. 9,278,123 discloses a solid composition comprising GLP-1 agonist semaglutide and sodium N-(8-(2-hydroxybenzoyl)amino)caprylic acid is in the range of 0.8-1.3 mmol which is also known as SNAC i.e. salcaprozate sodium, has been added to the formulation to enhance the bioavailability of semaglutide. U.S. Pat. No. 10,278,923 discloses method for treating diabetes and/or obesity in a subject with composition comprising GLP-1 peptide semaglutide and the enhancer is sodium N-(8-(2-hydroxybenzoyl)amino)caprylic acid (SNAC). U.S. Pat. No. 10,335,369 discloses a process of producing by dry granulation a granule comprising sodium N-(8-(2-hydroxybenzoyl)amino)caprylic acid and a lubricant, further mentions composition of the invention comprises a GLP-1 peptide. U.S. Pat. No. 10,933,120 discloses composition comprising first type of granules comprising sodium N-(8-(2-hydroxybenzoyl)amino)caprylic acid (SNAC) at least 75% (w/w) and no GLP-1 peptide and second type of granules comprising GLP-1 peptide i.e. semaglutide and no SNAC. U.S. Pat. No. 8,492,330 discloses a formulation comprising a GLP-1 compound and a delivery agent N-(8-(2-hydroxybenzoyl)amino)caprylic acid (NAC). U.S. Pat. No. 9,993,430 discloses a tablet comprising a granulate wherein said granulate comprises i) no more than 15% (w/w) semaglutide, and ii) at least 50% (w/w) salt of N-(8-(2-hydroxybenzoyl)amino)caprylic acid (NAC). U.S. Patent application 2020/000728 discloses a pharmaceutical composition comprising 0.5-50 mg of GLP-1 agonist and 20-800 mg of a salt of N-(8-(2-hydroxybenzoyl)amino)caprylic acid (NAC). WO 2019/215063 discloses a composition comprising GLP-1 agonist, a salt of N-(8-(2-hydroxybenzoyl)amino)caprylic acid (NAC) and a hydrotrope, wherein the hydrotrope is capable of increasing the solubility of SNAC at least 5-fold or such as at least 10-fold. WO 2010/020978 discloses an oral pharmaceutical composition comprising a protein a protease inhibitor, and an absorption enhancer such as N-(8-[2-hydroxybenzoyl)amino)caprylate (SNAC).

The aforementioned prior art formulations of GLP-1 agonists and/or semaglutide specifically comprising N-(8-(2-hydroxybenzoyl)amino)caprylate (NAC) and its sodium salt (SNAC) to enhance the bioavailability of GLP-1 agonists and/or semaglutide. Further the currently marketed semaglutide oral formulation with sodium N-(8-(2-hydroxybenzoyl)amino)caprylate (SNAC) i.e. Salcaprozate sodium exhibits improved stability. However, Salcaprozate sodium has the tendency of increasing water content during the storage which is evident from its stability studies, further manufacturing of Salcaprozate sodium is a very complex process. Hence, there still exists a need for developing the more stable oral formulations comprising GLP-1 agonists and/or Semaglutide that are commercially viable and easy to manufacture.

Therefore, the inventors of the present invention have surprisingly found that stable optimized semaglutide oral formulation formed by using permeation enhancers other than sodium N-(8-(2-hydroxybenzoyl)amino)caprylate (SNAC), exhibiting improved bioavailability of semaglutide. Further, the inventors of the present invention have also found that the excipients and the process of preparation used in the present invention stabilizes the formulation, provides rapid drug release, improves bioavailability and alleviates the limitations in the art by providing commercially viable semaglutide oral preparations with less degradation.

### SUMMARY OF INVENTION

Aspects of the present invention relates to stable pharmaceutical formulations comprising peptide or protein drug, permeation enhancers other than sodium N-(8-(2-hydroxybenzoyl)amino)caprylate (SNAC) and optionally one or more excipients selected from diluent, disintegrant, binder, lubricant, alkalizing agent, water soluble agent and anti-oxidant or combinations thereof.

In an another aspect, the present invention relates to stable pharmaceutical formulations comprising GLP-1 receptor agonist, permeation enhancers other than sodium N-(8-(2-hydroxybenzoyl)amino)caprylate (SNAC) and optionally one or more excipients selected from diluent, disintegrant, binder, lubricant, alkalizing agent, water soluble agent and anti-oxidant or combinations thereof.

In an another aspect, the present invention relates to stable pharmaceutical formulations comprising semaglutide, permeation enhancers other than sodium N-(8-(2-hydroxybenzoyl)amino)caprylate (SNAC) and optionally one or more excipients selected from diluent, disintegrant, binder, lubricant, alkalizing agent, water soluble agent and anti-oxidant or combinations thereof. The formulation according to the invention in an embodiment includes a very high content of permeation enhancer other than sodium N-(8-(2-hydroxybenzoyl)amino)caprylate (SNAC) and minimal content of further excipients as described herein below. The said formulation provides accelerated absorption, enabling fast and efficient uptake of semaglutide.

In an another aspect, the present invention relates to a formulation wherein the weight ratio of the permeation enhancers relative to the total composition, or in particular, relative to the other excipients of the composition, is very high.

In an another aspect, the present invention relates to a formulation comprising a peptide or protein drug, permeation enhancers comprising 40% to 98% w/w of the composition and one or more other excipients comprising 1% to 60% w/w of the composition.

In an another aspect, the invention relates to a formulation comprising a GLP-1 receptor agonist, permeation enhancers comprising 60% to 98% w/w of the composition and one or more other excipients comprising 1% to 30% w/w of the composition.

In an another aspect, the invention relates to a formulation comprising a semaglutide, permeation enhancers comprising 60% to 98% w/w of the composition and one or more other excipients comprising 1% to 30% w/w of the composition.

In an another aspect, the invention relates to a formulation comprising a GLP-1 receptor agonist, permeation enhancers comprising soyabean lecithin, arginine, lysine, phenyl alanine, tryptophan, sodium caprylate, sodium caprate, capsaicin analogue i.e. 10-Hydroxy-N-[(4-hydroxy-3-methoxyphenyl)methyl]decanamide (Compound 14), sodium laurate, sodium chenodeoxycholate, propyl gallate, dodecyl maltoside, tridecyl maltoside, tetradecyl maltoside, sucrose mono-dodecanoate, sucrose mono-tridecanoate, sucrose mono-tetradecanoate, sodium dodecyl sulfate, sodium hexanoate, sodium heptanoate, sodium octanoate, sodium nonanoate, sodium decanoate, sodium undecanoate, sodium dodecanoate, sodium tridecanoate, sodium tetradecanoate and combinations thereof comprising 70% to 98% w/w of the composition and one or more other excipients comprising 2% to 30% w/w of the composition.

In an another aspect, the invention relates to a formulation comprising a semaglutide, permeation enhancers comprising soyabean lecithin, arginine, lysine, phenyl alanine, tryptophan, sodium caprylate, sodium caprate, capsaicin analogue i.e. 10-Hydroxy-N-[(4-hydroxy-3-methoxyphenyl)methyl]decanamide (Compound 14), sodium laurate, sodium chenodeoxycholate, propyl gallate, dodecyl maltoside, tridecyl maltoside, tetradecyl maltoside, sucrose mono-dodecanoate, sucrose mono-tridecanoate, sucrose mono-tetradecanoate, sodium dodecyl sulfate, sodium hexanoate, sodium heptanoate, sodium octanoate, sodium nonanoate, sodium decanoate, sodium undecanoate, sodium dodecanoate, sodium tridecanoate, sodium tetradecanoate and combinations thereof comprising 70% to 98% w/w of the composition and one or more other excipients comprising 2% to 30% w/w of the composition.

In an another aspect, the invention relates to a formulation comprising a semaglutide, permeation enhancers comprising soyabean lecithin, arginine, lysine, phenyl alanine, tryptophan, sodium caprylate, sodium caprate, capsaicin analogue i.e. 10-Hydroxy-N-[(4-hydroxy-3-methoxyphenyl)methyl]decanamide (Compound 14), sodium laurate, sodium chenodeoxycholate, propyl gallate, dodecyl maltoside, tridecyl maltoside, tetradecyl maltoside, sucrose mono-dodecanoate, sucrose mono-tridecanoate, sucrose mono-tetradecanoate, sodium dodecyl sulfate, sodium hexanoate, sodium heptanoate, sodium octanoate, sodium nonanoate, sodium decanoate, sodium undecanoate, sodium dodecanoate, sodium tridecanoate, sodium tetradecanoate and combinations thereof comprising at least 70%, at least 80%, such as at least 90% w/w of the excipients of the composition.

In an another aspect, the invention relates to a method of preparing a pharmaceutical formulation as described herein such as a method comprising the steps of;
a) granulating a mixture comprising peptide or protein drug, permeation enhancers and optionally other excipients such as diluent, disintegrant, binder, lubricant, alkalizing agent, water soluble agent and anti-oxidant or combinations thereof.
b) compressing the granulate obtained in step a) into tablets and optionally adding further diluent and/or lubricant to the granulate prior to compression.

In an another aspect, the invention relates to a method of preparing a pharmaceutical formulation as described herein such as a method comprising the steps of;
a) granulating a mixture comprising GLP-1 receptor agonist, permeation enhancers and optionally other excipients such as diluent, disintegrant, binder, lubricant, alkalizing agent, water soluble agent and anti-oxidant or combinations thereof.
b) compressing the granulate obtained in step a) into tablets and optionally adding further diluent and/or lubricant to the granulate prior to compression.

In an another aspect, the invention relates to a method of preparing a pharmaceutical formulation as described herein such as a method comprising the steps of;
a) granulating a mixture comprising semaglutide, permeation enhancers and optionally other excipients such as diluent, disintegrant, binder, lubricant, alkalizing agent, water soluble agent and anti-oxidant or combinations thereof.
b) compressing the granulate obtained in step a) into tablets and optionally adding further diluent and/or lubricant to the granulate prior to compression.

In an another aspect, the invention relates to a process for the preparation of stable pharmaceutical formulation of semaglutide comprising;
a. co-sifting and mixing semaglutide, permeation enhancers and other excipients,
b. preparing granules using wet granulation, and
c. adding other excipients and compressing into tablet.

In an another aspect, the invention relates to a process for the preparation of stable pharmaceutical formulation of semaglutide comprising;
a. co-sifting and mixing semaglutide, permeation enhancers and other excipients,
b. preparing binder solution by dissolving binder in the water,
c. granulating the mixture of step (a) with binder solution of step (b),
d. drying the granules of step (c),
e. adding the diluent and lubricant to blend of step (d) and compressing into tablet.

In an another aspect, the invention relates to a formulation or a granule as defined herein for use in medicine, such as for treatment of diabetes or obesity, wherein said formulation is administered orally.

In a further aspect, the invention relates to a method of treating diabetes or obesity comprising administering the formulation as defined herein to a patient in need thereof, wherein said formulation is a tablet and is administered orally.

### DETAILED DESCRIPTION OF THE INVENTION

An aspect of the invention relates to a formulation comprising a peptide or protein drug and a permeation enhancer. The formulation may be in the form suitable for oral administration, such as a tablet, sachet or capsule.

An aspect of the invention relates to a formulation comprising a GLP-1 receptor agonist and a permeation enhancer. The formulation may be in the form suitable for oral administration, such as a tablet, sachet or capsule. In an embodiment the composition is an oral formulation, or a pharmaceutical formulation, such as an oral pharmaceutical formulation.

In embodiments, the invention relates to a formulation comprising a semaglutide and a permeation enhancer. The formulation may be in the form suitable for oral administration, such as a tablet, sachet or capsule. In an embodiment the composition is an oral formulation, or a pharmaceutical formulation, such as an oral pharmaceutical formulation.

In embodiments, the formulation according to the invention includes a high content of the permeation enhancer and a minimal content of further excipients as described herein below. The provided compositions display an accelerated dissolution and absorption, enabling fast and efficient uptake of the active pharmaceutical ingredient.

In embodiments, the term “peptide or protein drug” as used herein refers to any peptide or protein that is suitable to be used as a medicament to treat type 2 diabetes as well as a variety of other conditions. For example, the peptide or protein drug may be a linear peptide or protein drug or a cyclic peptide or protein drug.

In embodiments, the term “GLP-1 receptor agonist” as used herein refers to a compound, which fully or partially activates the human GLP-1 receptor. The term is thus equal to the term “GLP-1 agonist” used in other documents.

In embodiments, the term “comprising” means that the various components, ingredients, or steps, conjointly employed in practicing the present invention. Accordingly, the term “comprising” encompasses the more restrictive terms “consisting essentially of” and “consisting of”.

In embodiments, as used herein, the term “permeation enhancer” refers to an agent whose function is to increase absorption by enhancing membrane permeation, rather than increasing solubility.

In embodiments, the pharmaceutical formulation provided herein comprises peptide or protein drug is preferably selected from insulin, insulin aspart, insulin glulisine, insulin glargine, insulin detemir, NPH insulin, insulin degludec, insulin analog (e.g., a long acting basal insulin analog or a protease stabilized long acting basal insulin analog; exemplary insulin analogs include, without limitation, insulin lispro, insulin PEGIispro, GLP-1, a GLP-1 analog (e.g., an acylated GLP-1 analog or a diacylated GLP-1 analog) or GLP-1 agonist (also referred to as “glucagon-like peptide-1 receptor agonist” or “GLP-1 receptor agonist”), a GLP-2 agonist or analog (e.g., teduglutide or elsiglutide), amylin, an amylin analog, pramlintide, a somatostatin analog (e.g., octreotide, lanreotide, or pasireotide), desmopressin (e.g.,

desmopressin acetate), calcitonin (e.g., calcitonin-salmon), parathyroid hormone (PTH) and parathyroid hormone-related protein (PTHrP).

In embodiments, the pharmaceutical formulation provided herein comprises GLP-1 receptor agonist is selected from semaglutide, liraglutide, exenatide, exendin-4, lixisenatide, taspoglutide, albiglutide, dulaglutide, langlenatide, beinaglutide, efpeglenatide, coumaglutide and pharmaceutically acceptable salts thereof.

In embodiments, the pharmaceutical formulation provided herein comprises permeation enhancers, which are included in formulations to improve the absorption of a pharmacologically active drug. Exemplary permeation enhancers are selected from, but not limited to soyabean lecithin, arginine, lysine, phenyl alanine, tryptophan, sodium caprylate, sodium caprate, capsaicin analogue i.e. 10-Hydroxy-N-[(4-hydroxy-3-methoxyphenyl)methyl]decanamide (Compound 14), sodium laurate, sodium chenodeoxycholate, propyl gallate, dodecyl maltoside, tridecyl maltoside, tetradecyl maltoside, sucrose mono-dodecanoate, sucrose mono-tridecanoate, sucrose mono-tetradecanoate, sodium dodecyl sulfate, sodium hexanoate, sodium heptanoate, sodium octanoate, sodium nonanoate, sodium decanoate, sodium undecanoate, sodium dodecanoate, sodium tridecanoate, sodium tetradecanoate and combinations thereof.

In embodiments, the pharmaceutical formulation provided herein comprises excipients which act as diluents. Certain non-limiting examples of diluents include microcrystalline cellulose, maltodextrin, sucrose, xylitol, lactose, dextrose, sorbitol, dextrates, lactitol, kaolin, mannitol, starlac, starch, modified starches, dibasic calcium phosphate, tribasic calcium phosphate, calcium carbonate, calcium sulfate, magnesium carbonate, magnesium oxide. Preferably the diluent is selected from silicified microcrystalline cellulose, maltodextrin, sucrose, Xylitol, microcrystalline cellulose (Avicel-PH-101; Avicel-PH-102), lactose monohydrate and combinations thereof.

In embodiments, the pharmaceutical formulation provided herein includes a binder which hold ingredients in the formulation together. Exemplary binders are selected from, but not limited to, cellulose and its derivatives including, ethyl cellulose, hydroxypropyl cellulose, hydroxypropyl methylcellulose, methylcellulose and hydroxyethyl cellulose, carboxymethyl cellulose; gelatin, liquid glucose; starch and its derivatives; hydrocolloids; sugars; polyvinyl pyrrolidone, sodium alginate, acacia, alginic acid, tragacanth, used either alone or combinations comprising one or more of the foregoing binders.

In embodiments, the pharmaceutical formulation provided herein includes disintegrant. Exemplary disintegrants are selected from, but not limited to, cellulose and its derivatives including low-substituted hydroxypropyl cellulose; cross-linked polyvinylpyrrolidone; polyvinylpyrrolidone; cross-linked sodium carboxymethylcellulose, sodium carboxymethylcellulose, microcrystalline cellulose; sodium starch glycolate; ion-exchange resins; starch and modified starches; formalin-casein; used either alone or combinations thereof

In embodiments, the pharmaceutical formulation provided herein includes a Lubricants and/or glidants aids in the processing of powder materials. Exemplary lubricants are selected from, but not limited to, calcium stearate, magnesium stearate, glycerol behenate, mineral oil, polyethylene glycol, sodium stearyl fumarate, stearic acid, talc, vegetable oil, and zinc stearate, used either alone or combinations thereof. Exemplary glidants include, but not limited to, talc, silicon dioxide, cornstarch and the like used either alone or in combination thereof.

In embodiments, the formulation provided herein includes anti-oxidants and alkalizing agents. Suitable anti-oxidants are selected from, but not limited to, butylated hydroxyanisole (BHA), butylated hydroxytoluene (BHT), sodium metabisulfite (SMB), propyl gallate (PG) and cysteine (CYS) and the like used either alone or in combination thereof. Suitable alkalizing agents are selected from, but not limited to, calcium carbonate, sodium bicarbonate, sodium citrate and potassium citrate and the like used either alone or in combination thereof.

In embodiments, the formulation provided herein includes water soluble agents. Exemplary water soluble agent or suitable combination of water soluble agents or solubilizers preferred in this invention include surface active agents such as sodium docusate, sodium lauryl sulfate, sodium stearyl fumarate, Tweens® and Spans (PEO modified sorbitan monoesters and fatty acid sorbitan esters), polyoxyethylene polypropyleneglycol, such as Poloxamer® 124, Poloxamer® 188, Poloxamer® 237, Poloxamer® 388, Poloxamer® 407, polyoxyethylene castor oil derivatives, polyoxyethylene alkyl ethers, polyoxyethylene alkylaryl ethers, polyethylene glycol fatty acid esters poly(ethylene oxide)-polypropylene oxide-poly(ethylene oxide) block copolymers (aka Pluronics™); complexing agents such as low molecular weight polyvinyl pyrrolidone and low molecular weight hydroxypropyl methyl cellulose; molecules that aid solubility by molecular entrapment such as cyclodextrins.

As shown in the examples herein, the formulations of the invention have a very high content of the permeation enhancer. This very high content can be defined relative to the full content of the tablets including also the active pharmaceutical ingredient or alternatively relative to the total content of excipients excluding the active pharmaceutical ingredient.

In embodiments, the pharmaceutical composition comprises peptide or protein drug and permeation enhancers comprising at least or above 70 w/w % of the composition.

In embodiments, the pharmaceutical composition comprises GLP-1 receptor agonist and permeation enhancers comprising at least or above 70 w/w % of the composition.

In embodiments, the pharmaceutical composition comprises semaglutide and permeation enhancers comprising at least or above 70 w/w % of the composition.

In embodiments, the permeation enhancers comprises at least 90 w/w %, such as at least 85 w/w %, such as at least 80 w/w %, such as at least 75 w/w %, such as at least 70 w/w % of the excipients of the composition.

In embodiments, the pharmaceutical formulation comprises
a) peptide or protein drug and
b) permeation enhancers comprising soyabean lecithin, arginine, lysine, phenyl alanine, tryptophan, sodium caprylate, sodium caprate, capsaicin analogue i.e. 10-Hydroxy-N-[(4-hydroxy-3-methoxyphenyl)methyl]decanamide (Compound 14), sodium laurate, sodium chenodeoxycholate, propyl gallate, dodecyl maltoside, tridecyl maltoside, tetradecyl maltoside, sucrose mono-dodecanoate, sucrose mono-tridecanoate, sucrose mono-tetradecanoate, sodium dodecyl sulfate, sodium hexanoate, sodium heptanoate, sodium octanoate, sodium nonanoate, sodium decanoate, sodium undecanoate, sodium dodecanoate, sodium tridecanoate, sodium tetradecanoate and combinations thereof and wherein said permeation enhancers comprises at least or above 70 w/w % of the composition.

In embodiments, the pharmaceutical formulation comprises
a) GLP-1 receptor agonist and
b) permeation enhancers comprising soyabean lecithin, arginine, lysine, phenyl alanine, tryptophan, sodium caprylate, sodium caprate, capsaicin analogue i.e. 10-Hydroxy-N-[(4-hydroxy-3-methoxyphenyl)methyl]decanamide (Compound 14), sodium laurate, sodium chenodeoxycholate, propyl gallate, dodecyl maltoside, tridecyl maltoside, tetradecyl maltoside, sucrose mono-dodecanoate, sucrose mono-tridecanoate, sucrose mono-tetradecanoate, sodium dodecyl sulfate, sodium hexanoate, sodium heptanoate, sodium octanoate, sodium nonanoate, sodium decanoate, sodium undecanoate, sodium dodecanoate, sodium tridecanoate, sodium tetradecanoate and combinations thereof and wherein said permeation enhancers comprises at least or above 70 w/w % of the composition.

In embodiments, the pharmaceutical formulation comprises
a) semaglutide and
b) permeation enhancers comprising soyabean lecithin, arginine, lysine, phenyl alanine, tryptophan, sodium caprylate, sodium caprate, capsaicin analogue i.e. 10-Hydroxy-N-[(4-hydroxy-3-methoxyphenyl)methyl]decanamide (Compound 14), sodium laurate, sodium chenodeoxycholate, propyl gallate, dodecyl maltoside, tridecyl maltoside, tetradecyl maltoside, sucrose mono-dodecanoate, sucrose mono-tridecanoate, sucrose mono-tetradecanoate, sodium dodecyl sulfate, sodium hexanoate, sodium heptanoate, sodium octanoate, sodium nonanoate, sodium decanoate, sodium undecanoate, sodium dodecanoate, sodium tridecanoate, sodium tetradecanoate and combinations thereof and wherein said permeation enhancers comprises at least or above 70 w/w % of the composition.

In embodiments the pharmaceutical formulation comprises
a) semaglutide and
b) excipients, wherein the excipients are
   i. permeation enhancers comprising soyabean lecithin, arginine, lysine, phenyl alanine, tryptophan, sodium caprylate, sodium caprate, capsaicin analogue i.e. 10-Hydroxy-N-[(4-hydroxy-3-methoxyphenyl)methyl]decanamide (Compound 14), sodium laurate, sodium chenodeoxycholate, propyl gallate, dodecyl maltoside, tridecyl maltoside, tetradecyl maltoside, sucrose mono-dodecanoate, sucrose mono-tridecanoate, sucrose mono-tetradecanoate, sodium dodecyl sulfate, sodium hexanoate, sodium heptanoate, sodium octanoate, sodium nonanoate, sodium decanoate, sodium undecanoate, sodium dodecanoate, sodium tridecanoate, sodium tetradecanoate and combinations thereof and
   ii. one or more further excipients.

In embodiments, the permeation enhancers comprising above 60 w/w %, such as above 70 w/w %, such as above 80 w/w % or such as above 90 w/w % of the composition.

In embodiments, the permeation enhancers comprising at least 65 w/w %, such as at least 75 w/w %, such as at least 85 w/w % or such as at least 95 w/w % of the excipients of the composition.

The pharmaceutical formulations according to the invention is preferably produced in a dosage form suitable for oral administration as described herein below. In the following the absolute amounts of the ingredients of the composition of the invention are provided with reference to the content in a dosage unit i.e. per tablet, capsule or sachet.

The pharmaceutical formulations of the invention may in a further embodiment comprise at most 400 mg of said permeation enhancers. In one embodiment the invention relates to a composition wherein a dose unit comprises at most 300 mg of said permeation enhancers.

In embodiments, the amount of permeation enhancer in the formulation is at least 20 mg, such as at least 25 mg, such as at least 50 mg, such as at least 75 mg, at least 100 mg, at least 125 mg, at least 150 mg, at least 175 mg, at least 200 mg, at least 250 mg, at least 300 mg, at least 350 mg and at least 400 mg per dose unit.

In embodiments, the amount of permeation enhancer in the formulation is in the range 50-500 mg, such as 50-400 mg, such as 75-400 mg, such as 80-350 mg or such as from around 100 to around 400 mg per dose unit.

In embodiments, the amount of permeation enhancer is in the range of 250-400 mg, such as 250-350 mg, such as 275-325 mg, such as around 300 mg per dose unit.

In embodiments, a dose unit of the pharmaceutical formulations of the invention comprises 0.1-100 mg of the peptide or protein drug.

In embodiments, a dose unit of the pharmaceutical formulations of the invention comprises 0.1-100 mg or 0.2 to 100 mg of the GLP-1 receptor agonist.

In embodiments, a dose unit of the pharmaceutical formulations of the invention comprises 0.1-100 mg or 0.2 to 100 mg of the semaglutide.

In embodiments, a dose unit of the formulations comprises an amount of semaglutide is in the range of 0.2 to 50 mg or 1 to 40 mg.

In embodiments, a dose unit comprises 2 to 20 mg of the semaglutide, such as 2-15 mg, such as 2, 3, 4, 5, 6 or 7 mg, such as 3 or 7 mg, or such as 8, 10, 12 or 14 mg, such as 14 mg or such as 20 mg of the semaglutide per dose unit.

The composition may be administered in several dosage forms, for example as a tablet; a coated tablet; a sachet or a capsule such as hard or soft shell capsules.

The composition may be in the form of a dose unit, such as a tablet. In some embodiments the weight of the unit dose is in the range of 50 mg to 1000 mg, such as in the range of 50-750 mg, or such as in the range of 100-500 mg.

In embodiments, the weight of the dose unit is in the range of 100 mg to 500 mg, such as in the range of 50-300 mg or such as in the range of 200-500 mg.

In embodiments, the present invention provides a stable pharmaceutical formulation of peptide or protein drug and a process for preparing the same by using wet granulation process.

In embodiments, the present invention provides a stable pharmaceutical formulation of GLP-1 receptor agonist and a process for preparing the same by using wet granulation process.

In embodiments, the present invention provides a stable pharmaceutical formulation of semaglutide and a process for preparing the same by using wet granulation process.

In embodiments the composition may be granulated prior to the compression. The composition may comprise an intragranular part and/or an extragranular part, wherein the intragranular part has been granulated and the extragranular part has been added after granulation.

The intragranular part may comprise semaglutide, the permeation enhancer and/or an excipient, such as a diluent, anti-oxidant, alkalizing agent and binder. In some embodiments the intragranular part comprises the permeation enhancer, diluent, anti-oxidant, alkalizing agent and binder.

In embodiments, the extragranular part comprises an excipient, such as a diluent, lubricant and/or glidant.

In embodiments, the present invention provides a process for the preparation of stable pharmaceutical formulation of peptide or protein drug comprising;
a. co-sifting and mixing peptide or protein drug, permeation enhancers and other excipients,
b. preparing granules using wet granulation, and
c. adding other excipients and compressing into tablet.

In embodiments, the present invention provides a process for the preparation of stable pharmaceutical formulation of GLP-1 receptor agonist comprising;
a. co-sifting and mixing GLP-1 receptor agonist, permeation enhancers and other excipients,
b. preparing granules using wet granulation, and
c. adding other excipients and compressing into tablet.

In some embodiments, the present invention provides a process for the preparation of stable pharmaceutical formulation of semaglutide comprising;
a. co-sifting and mixing semaglutide, permeation enhancers and other excipients,
b. preparing granules using wet granulation, and
c. adding other excipients and compressing into tablet.

In embodiments, the present invention provides a process for the preparation of stable pharmaceutical formulation of semaglutide comprising;
a. co-sifting and mixing semaglutide, permeation enhancers and other excipients,
b. preparing binder solution by dissolving binder in the water,
c. granulating the mixture of step (a) with binder solution of step (b),
d. drying the granules of step (c),
e. adding the diluent and lubricant to blend of step (d) and compressing into tablet.

The invention now will be described in particularity with the following illustrative examples; however, the scope of the present invention is not intended to be, and shall not be, limited to the exemplified embodiments below:

### EXAMPLES

### Example 1

| | | 3 mg strength | 7 mg strength | 14 mg strength |
|---|---|---|---|---|
| Sl. No. | Ingredients | mg | mg | mg |
| 1 | Semaglutide | 3.00 | 7.00 | 14.00 |
| 2 | Arginine | 50.00 | 50.00 | 50.00 |
| 3 | Sodium | 100.00-150.00 | 100.00-150.00 | 100.00-150.00 |
| | caprylate | | | |
| 4 | Povidone | 4.00 | 4.00 | 4.00 |
| 5 | Microcrys- | 216.00 | 216.00 | 216.00 |
| | talline | | | |
| | cellulose | | | |
| 6 | Mag. | 2.00 | 2.00 | 2.00 |
| | Stearate | | | |

| | | | |
|---|---|---|---|
| Total weight (mg) | 400.00 | 400.00 | 400.00 |

### Manufacturing Process:

1. Co-Sift all the raw materials from 1 to 5 through 600 μm mesh (ASTM, #30 sieve).
2. Mix material from step 1 in Rapid mixer granulator for 10 minutes with Impeller and Chopper at slow speed.
3. Add purified water to step 2 with impeller and chopper at slow speed.
4. Dry the material of step 3 in Rapid dryer and mill using quadro co-mill using suitable screen.
5. Blend the milled materials of step 4 in a blender for 15 minutes.
6. Sift Magnesium stearate through ASTM #60 and add to the above step.
7. Lubricate the blend in a suitable blender for 5 minutes.
8. Compress the blend of step 7 into tablets using multi station compression machine.

### Example 2

| | | 20 mg strength |
|---|---|---|
| Sl. No. | Ingredients | mg |
| 1 | Octreotide | 20.00 |
| 2 | Tryptophan | 250.00 |
| 3 | Sodium caprylate | 100.00 |
| 4 | Povidone | 4.00 |
| 5 | Microcrystalline cellulose | 34.00 |
| 6 | Mag. Stearate | 2.00 |

| | |
|---|---|
| Total weight (mg) | 410.00 |

### Manufacturing Process:

1. Co-Sift all the raw materials from 1 to 5 through 600 μm mesh (ASTM, #30 sieve).
2. Mix material from step 1 in Rapid mixer granulator for 10 minutes with Impeller and Chopper at slow speed.
3. Add purified water to step 2 with impeller and chopper at slow speed.
4. Dry the material of step 3 in Rapid dryer and mill using quadro co-mill using suitable screen.
5. Blend the milled materials of step 4 in a blender for 15 minutes.
6. Sift Magnesium stearate through ASTM #60 and add to the above step.
7. Lubricate the blend in a suitable blender for 5 minutes.
8. Fill the granules into capsule shells.

### Example 3

| | | 25 mg strength |
|---|---|---|
| Sl. No. | Ingredients | mg |
| 1 | Liraglutide | 25.00 |
| 2 | Tryptophan | 150.00 |
| 3 | Sodium caprate | 150.00 |
| 4 | Povidone | 4.00 |
| 5 | Microcrystalline cellulose | 84.00 |
| 6 | Mag. Stearate | 2.00 |

| | |
|---|---|
| Total weight (mg) | 410.00 |

### Manufacturing Process:

1. Co-Sift all the raw materials from 1 to 5 through 600 μm mesh (ASTM, #30 sieve).
2. Mix material from step 1 in Rapid mixer granulator for 10 minutes with Impeller and Chopper at slow speed.
3. Add purified water to step 2 with impeller and chopper at slow speed.
4. Dry the material of step 3 in Rapid dryer and mill using quadro co-mill using suitable screen.
5. Blend the milled materials of step 4 in a blender for 15 minutes.
6. Sift Magnesium stearate through ASTM #60 and add to the above step.
7. Lubricate the blend in a suitable blender for 5 minutes.
8. Fill the granules into sachets.

### Example 4

| | | 25 mg strength |
|---|---|---|
| Sl. No. | Ingredients | mg |
| 1 | Insulin (in IU) | 5.00 |
| 2 | Tryptophan | 150.00 |
| 3 | Arginine | 100.00 |
| 4 | Sodium caprate | 150.00 |
| 5 | Povidone | 4.00 |
| 6 | Microcrystalline cellulose | 89.00 |
| 7 | Mag. Stearate | 2.00 |

| | |
|---|---|
| Total weight (mg) | 500.00 |

### Manufacturing Process:

1. Co-Sift all the raw materials from 1 to 6 through 600 μm mesh (ASTM, #30 sieve).
2. Mix material from step 1 in Rapid mixer granulator for 10 minutes with Impeller and Chopper at slow speed.
3. Add purified water to step 2 with impeller and chopper at slow speed.
4. Dry the material of step 3 in Rapid dryer and mill using quadro co-mill using suitable screen.
5. Blend the milled materials of step 4 in a blender for 15 minutes.
6. Sift Magnesium stearate through ASTM #60 and add to the above step.
7. Lubricate the blend in a suitable blender for 5 minutes.
8. Fill the powder into capsule shells.

### Example 5

| Sr. No. | Ingredients | Quantity/Tablet |
|---|---|---|
| 1 | Semaglutide | 14.000 |
| 2 | L-Arginine | 50.000 |
| 3 | Sodium Caprylate | 250.000 |
| 4 | Povidone | 10.000 |
| 5 | Microcrystallince cellullose PH-102 | 72.000 |
| 6 | Magnesium Stearate | 4.000 |

| | |
|---|---|
| Total weight (mg) | 400.00 |

### Manufacturing Process:

1. Co-Sift all the raw materials from 1 to 5 through 600 μm mesh (ASTM, #30 sieve).
2. Mix material from step 1 in Rapid mixer granulator for 10 minutes with Impeller and Chopper at slow speed.
3. Add purified water to step 2 with impeller and chopper at slow speed.
4. Dry the material of step 3 in Rapid dryer and mill using quadro co-mill using suitable screen.
5. Blend the milled materials of step 4 in a blender for 15 minutes.
6. Sift Magnesium stearate through ASTM #60 and add to the above step.
7. Lubricate the blend in a suitable blender for 5 minutes.
8. Fill the powder into capsule shells.

### Comparison of In-Vitro Dissolution Profile of Example 5 Composition with Reference Product

The capsules of Semaglutide prepared as per the compositions of example 5 and reference product Rybelsus® (Semaglutide) Tablets were subjected to dissolution studies in 500 ml of Phosphate buffer pH 6.8 with 0.05% Brij 35 at 37° C. using USP apparatus II with paddle speed at 70 rpm and the data is shown in table 1.

TABLE 1

| | Semaglutide | Rybelsus ® |
|---|---|---|
| Time Points | Capsules 14 mg | (Semaglutide) |
| [Hrs] | (Example 5) | Tablets 14 mg |
| 10 | 78 | 54 |
| 15 | 89 | 73 |
| 30 | 94 | 95 |
| 45 | 96 | 95 |

The stability of Semaglutide capsules prepared as per the compositions of example 5 was evaluated at 40° C./75% RH [3 months] storage condition by various parameters such as assay and related substances including β-Asp9-semaglutide, Plus-Gly4-semaglutide, D-His 1-Semaglutide and plus-Gly 16-semaglutide. The below table 2 shows the stability of Semaglutide capsules of Example 5 at initial and upon storage periods at 40° C./75% relative humidity and table 3 shows the dissolution profile example 5 after stability studies at 40° C./75% relative humidity for 3 months.

TABLE 2

| Parameters | | Control | 40° C./75% RH-3M |
|---|---|---|---|
| Assay | | 103 | 103.4 |
| Related | β-Asp9-semaglutide | ND | ND |
| substances/ | Plus-Gly4-semaglutide | 0.02 | 0.08 |
| impurities | D-His 1-Semaglutide | ND | 0.09 |
| | plus-Gly 16- | ND | ND |
| | semaglutide | | |
| | Unknown | 0.19 | 0.15 |
| | Total | 0.35 | 0.42 |

TABLE 3

| | |
|---|---|
| Time Points | Semaglutide Capsules 14 mg (Example 5) |

| [Hrs] | Initial | 40° C./75% RH-3M |
|---|---|---|
| 10 | 78 | 88 |
| 15 | 89 | 94 |
| 30 | 94 | 98 |
| 45 | 96 | 100 |

### Example 6

| Sr. No. | Ingredients | Quantity/Tablet |
|---|---|---|
| 1 | Octreotide | 20.000 |
| 2 | L-Arginine | 50.000 |
| 3 | Compound 14 | 300.000 |
| 4 | Povidone | 10.000 |
| 5 | Microcrystalline cellulose | 76.000 |
| 6 | Magnesium Stearate | 4.000 |

| | |
|---|---|
| Total weight (mg) | 460.00 |

### Manufacturing Process:

1. Co-Sift all the raw materials from 1 to 5 through 600 μm mesh (ASTM, #30 sieve).
2. Mix material from step 1 in Rapid mixer granulator for 10 minutes with Impeller and Chopper at slow speed.
3. Add purified water to step 2 with impeller and chopper at slow speed.
4. Dry the material of step 3 in Rapid dryer and mill using quadro co-mill using suitable screen.
5. Blend the milled materials of step 4 in a blender for 15 minutes.
6. Sift Magnesium stearate through ASTM #60 and add to the above step.
7. Lubricate the blend in a suitable blender for 5 minutes.
8. Fill the powder into capsule shells.

### Example 7

| | | |
|---|---|---|
| Sr. No. | Ingredients | Quantity/Tablet |

Intragranular

| | | |
|---|---|---|
| 1 | Semaglutide | 14.000 |
| 2 | Tryptophan | 250.000 |
| 3 | Compound 14 | 150.000 |
| 4 | Povidone | 10.000 |
| 5 | Microcrystalline cellulose | 72.000 |
| 6 | Magnesium Stearate | 4.000 |

| | |
|---|---|
| Total weight (mg) | 500.00 |

### Manufacturing Process:

1. Co-Sift all the raw materials from 1 to 5 through 600 μm mesh (ASTM, #30 sieve).
2. Mix material from step 1 in Rapid mixer granulator for 10 minutes with Impeller and Chopper at slow speed.
3. Add purified water to step 2 with impeller and chopper at slow speed.
4. Dry the material of step 3 in Rapid dryer and mill using quadro co-mill using suitable screen.
5. Blend the milled materials of step 4 in a blender for 15 minutes.
6. Sift Magnesium stearate through ASTM #60 and add to the above step.
7. Lubricate the blend in a suitable blender for 5 minutes.
8. Compress the blend into tablets.

We claim:

1. A stable pharmaceutical formulation comprising peptide or protein drug and permeation enhancers other than sodium N-(8-(2-hydroxybenzoyl) amino)caprylate (SNAC).

2. The stable pharmaceutical formulation as claimed in claim 1, wherein the peptide or protein drug is selected from the group comprising insulin, insulin aspart, insulin glulisine, insulin glargine, insulin detemir, NPH insulin, insulin degludec, insulin analog (e.g., a long acting basal insulin analog or a protease stabilized long acting basal insulin analog; exemplary insulin analogs include, without limitation, insulin lispro, insulin PEGIispro, GLP-1, a GLP-1 analog (e.g., an acylated GLP-1 analog or a diacylated GLP-1 analog) or GLP-1 agonist (also referred to as “glucagon-like peptide-1 receptor agonist” or “GLP-1 receptor agonist”), a GLP-2 agonist or analog (e.g., teduglutide or elsiglutide), amylin, an amylin analog, pramlintide, a somatostatin analog (e.g., octreotide, lanreotide, or pasireotide), desmopressin (e.g., desmopressin acetate), calcitonin (e.g., calcitonin-salmon), parathyroid hormone (PTH) and parathyroid hormone-related protein (PTHrP).

3. The stable pharmaceutical formulation as claimed in claim 1, wherein the permeation enhancers are selected from but not limited to soyabean lecithin, arginine, lysine, phenyl alanine, tryptophan, sodium caprylate, sodium caprate, capsaicin analogue 10-Hydroxy-N-[(4-hydroxy-3-methoxyphenyl) methyl] decanamide (Compound 14), sodium laurate, sodium chenodeoxycholate, propyl gallate, dodecyl maltoside, tridecyl maltoside, tetradecyl maltoside, sucrose mono-dodecanoate, sucrose mono-tridecanoate, sucrose mono-tetradecanoate, sodium dodecyl sulfate, sodium hexanoate, sodium heptanoate, sodium octanoate, sodium nonanoate, sodium decanoate, sodium undecanoate, sodium dodecanoate, sodium tridecanoate, sodium tetradecanoate and combinations thereof.

4. The stable pharmaceutical formulation as claimed in claim 2, wherein the GLP-1 receptor agonist is selected from the group comprising semaglutide, liraglutide, exenatide, exendin-4, lixisenatide, taspoglutide, albiglutide, dulaglutide, langlenatide, beinaglutide, efpeglenatide, coumaglutide and pharmaceutically acceptable salts thereof.

5. The stable pharmaceutical formulation as claimed in claim 1, wherein said formulation further comprises one or more excipients selected from diluent, disintegrant, binder, lubricant, alkalizing agent and anti-oxidant or combinations thereof.

6. The stable pharmaceutical formulation as claimed in claim 1, wherein said formulation comprising a GLP-1 receptor agonist, permeation enhancers other than sodium N-(8-(2-hydroxybenzoyl)amino)caprylate (SNAC) comprising 40% to 98% w/w of the composition and one or more other excipients comprising 1% to 30% w/w of the composition.

7. A method of preparing stable pharmaceutical formulation as claimed in claim 6, wherein said method comprising the steps of;
a) granulating a mixture comprising GLP-1 receptor agonist, permeation enhancers and other excipients such as diluent, disintegrant, binder, lubricant, alkalizing agent and anti-oxidant or combinations thereof.
b) compressing the granulate obtained in step a) into tablets and optionally adding further diluent and/or lubricant to the granulate prior to compression.

8. A stable pharmaceutical formulation comprising semaglutide, permeation enhancers other than sodium N-(8-(2-hydroxybenzoyl)amino)caprylate (SNAC) comprising 40% to 98% w/w of the composition and one or more other excipients comprising 1% to 30% w/w of the composition.

9. The stable pharmaceutical formulation as claimed in claim 8, wherein the pharmaceutical formulation comprises
a) semaglutide and
b) permeation enhancers selected from the group comprising soyabean lecithin, arginine, lysine, phenyl alanine, tryptophan, sodium caprylate, sodium caprate, capsaicin analogue 10-Hydroxy-N-[(4-hydroxy-3-methoxyphenyl) methyl] decanamide (Compound 14), sodium laurate, sodium chenodeoxycholate, propyl gallate, dodecyl maltoside, tridecyl maltoside, tetradecyl maltoside, sucrose mono-dodecanoate, sucrose mono-tridecanoate, sucrose mono-tetradecanoate, sodium dodecyl sulfate, sodium hexanoate, sodium heptanoate, sodium octanoate, sodium nonanoate, sodium decanoate, sodium undecanoate, sodium dodecanoate, sodium tridecanoate, sodium tetradecanoate and combinations thereof and wherein said permeation enhancers comprises at least or above 70 w/w % of the composition.

10. A process for the preparation of stable pharmaceutical formulation of semaglutide as claimed in claim 8, wherein said process comprising;
a. co-sifting and mixing semaglutide, permeation enhancers and other excipients,
b. preparing binder solution by dissolving binder in the water,
c. granulating the mixture of step (a) with binder solution of step (b),
d. drying the granules of step (c),
e. adding the diluent and lubricant to blend of step (d) and compressing into tablet.
